(19) United States
(12) Patent Application Publication
(10) Pub. No.: US 2023/0164496 A1
(43) Pub. Date: May 25, 2023

(54) METHOD OF OPERATING A HEARING INSTRUMENT

(71) Applicant: Sivantos Pte. Ltd., Singapore (SG)

(72) Inventors: Maja Serman, Erlangen (DE); Cecil Wilson, Erlangen (DE); Ulrich Giese, Erlangen (DE)

(21) Appl. No.: 17/993,146

(22) Filed: Nov. 23, 2022

(30) Foreign Application Priority Data
Nov. 25, 2021 (DE) 10 2021 213 300.2

(51) Int. Cl. H04R 25/00; G10L 25/63; A61B 5/16; A61B 5/00

(52) U.S. Cl. CPC H04R 25/505; G10L 25/63; H04R 25/75; A61B 5/165; A61B 5/4836; A61B 5/6817; H04R 2225/41; H04R 2225/61; H04R 2225/43; H04R 2430/01; A61B 5/02416

(57) ABSTRACT

A method for operating a hearing instrument, wherein a biometric parameter of a wearer of the hearing instrument is acquired by a sensor of the hearing instrument and/or an electrical input signal is generated from an ambient sound by at least one microphone of the hearing instrument, and the presence of a specific ambient acoustic situation is detected from a plurality of different ambient acoustic situations by an analysis of the input signal, based on the acquired biometric parameter or the detected ambient acoustic situation a critical stress level of the wearer is determined, and then, in accordance with the critical stress level thus determined, a parameter of a signal processor of the hearing instrument is adjusted to a predefined parameter value.

The invention relates to a method for operating a hearing instrument and to a hearing aid system having a hearing instrument.

Hearing impairments can have a variety of causes. Thus, on the one hand in a so-called sound conduction hearing loss, there is a substantially mechanical inhibition of the outer ear or the organs of the middle ear so that a sound entering a person's ear canal is not sufficiently well transmitted to the nerve cells of the inner ear. On the other hand, in the case of so-called sensorineural hearing loss, these nerve cells or also the auditory nerve may be damaged. Combinations of the two mentioned forms of hearing loss are also possible.

In the sensorineural form in particular, due to the cause of the hearing loss originating in the nerves, a general health or general well-being of a hearing-impaired person can also affect the extent to which the hearing loss is manifested, depending on the degree of loss.

Conversely, the general well-being of a hearing-impaired person can be impaired if a particularly high hearing effort has to be made, for example, in order to be able to understand a conversation partner at all.

The object of the invention is therefore to specify a method by means of which a general well-being of a wearer of the hearing instrument can be taken into account in the operation of a hearing instrument in a manner that is most informative to the individual. A further object of the invention is to specify a hearing system which comprises a hearing instrument and which enables the aforementioned consideration of the general well-being of the wearer.

The first-mentioned object is achieved according to the invention by a method for operating a hearing instrument, wherein a biometric parameter of a wearer of the hearing instrument is acquired by means of a sensor, in particular a sensor of the hearing instrument, and/or an electrical input signal is generated from an ambient sound by means of at least one microphone of the hearing instrument, and the presence of a specific ambient acoustic situation is detected from a plurality of different ambient acoustic situations by means of an analysis of the input signal, based on the acquired biometric parameter or the detected ambient acoustic situation a critical stress level of the wearer is determined, and then, depending on the critical stress level thus determined, at least one parameter of a signal processing of the hearing instrument is adjusted to a predefined parameter value. Advantageous embodiments, some of which are inventive in themselves, are set out in the dependent claims and in the following description.

A hearing instrument in this case includes in general any apparatus which is designed to generate a sound signal from an electrical signal—which can also be given by an internal signal of the device—and to supply said signal to the hearing of a wearer of this apparatus, in particular a set of headphones (e.g. in the form of an “earplug”), a headset, data glasses with loudspeaker, etc. A hearing instrument also includes, however, a hearing device in the narrower sense, i.e. a device which is designed to treat a hearing impairment of the wearer (as a main or a secondary function), and in which an input signal generated from an ambient sound by means of a microphone is processed to form an output signal and is amplified in particular in a frequency-band-dependent manner, and an output sound signal generated from the output signal by means of a loudspeaker or similar is suitable for at least partially compensating the hearing impairment of the wearer (in particular in a user-specific manner).

The at least one parameter value is preferably defined in advance such that a processing of an internal signal—i.e., for example, in the case of a hearing aid an input signal, as well as generally any output signal which is to be converted into an output sound signal by a loudspeaker etc. of the hearing instrument—with the parameter value for said parameter of the signal processing is provided for, and also suitable for, reducing the stress level of the wearer. This can be carried out, for example, on the basis of psychoacoustic empirical values for the parameter, on the basis of general medical knowledge relating to the development and reduction of stress and the influence of sound on it, or else by an individual tuning of the parameter by a hearing aid acoustician or similar.

The biometric parameter detected by means of the sensor is preferably of such a type that a stress level of the wearer can be inferred from the biometric parameter, i.e. in particular a correlation, preferably a monotonic relationship, exists between the biometric parameter and the stress level. A critical stress level can also be defined directly via the biometric parameter: if the biometric parameter is above a critical value, biomedical considerations are used to infer that a critical stress level has been reached.

In particular, a characteristic parameter of a cardiovascular activity of the wearer is acquired as a biometric parameter. One such characteristic parameter of a cardiovascular activity of the wearer can be preferably provided in the form of a heart rate of the wearer and/or a rate of change of the heart rate and/or a systolic and/or diastolic blood pressure of the wearer and/or a duration of an exertion phase of the heart. In general, the quantities mentioned show a particularly high correlation with a subjectively perceived stress level.

The heart rate or the systolic or diastolic blood pressure is preferably measured by means of photoplethysmography (PPG). In this case, the sensor is provided by a PPG sensor. The rate of change in the heart rate can then be derived directly from the heart rate. A PPG sensor can be easily integrated directly into the hearing instrument. However, the heart rate or the systolic or diastolic blood pressure can in particular also be measured by means of an ear-canal microphone, which is directed into an ear canal of the wearer when the hearing instrument is worn in the proper manner. The ear-canal microphone may be arranged in particular in or on an earpiece which the wearer at least partially inserts into the entrance of the ear canal to wear the hearing instrument in the proper manner. The ear-canal microphone is preferably designed to also perform other functions of the hearing instrument, e.g. for suppressing an acoustic feedback on the hearing instrument and/or suppressing an occlusion of the ear canal by the hearing instrument. The rate of change in the heart rate can be derived directly from the heart rate, in particular.

An ambient acoustic situation, which can in particular also be referred to as a listening situation, is preferably to be understood as a process of combining potentially different individual events of the ambient sound into a group, wherein for the combination one or more acoustic parameters such as, for example, frequency-band-dependent absolute and relative sound levels, noise levels, noise backgrounds etc. are evaluated, and two individual events are combined into the same group, i.e. assigned to the same ambient acoustic situation, if the evaluated parameters are sufficiently similar. This can be carried out, for example, over appropriate intervals for the evaluated parameters and/or a vectorial listing of the parameters evaluated in each case, followed by determination of a vector distance (e.g. from a “base vector” for an ambient acoustic situation).

Thus, the said analysis determines which of the various predetermined ambient acoustic situations the individual event caused by a current ambient sound is to be assigned to.

In particular, the normal operation of the hearing instrument may include a continuous learning, in such a way that individual ranges of values for the biometric parameter for detecting the critical stress level, individual acoustic environmental situations that can cause a critical stress level, and/or individual ranges of values of the signal processing parameter to reduce the stress level, can be captured and “learned” for future use.

The signal processing with a parameter value that is intended for and suitable for reducing the stress level of the wearer has the following background:

from a psychological point of view, stress usually means a response to various events that can result from a person's relationship to their immediate environment or surroundings. Stress can be acute or chronic, wherein in addition to the usual meaning of “negative” stress (“distress”) there can also be positive “eustress”, and whichever one occurs depends on the exact circumstances.

The ability of the human brain to alter or even suppress sensitivity to sensory stimuli, which may be irrelevant or stress-causing, is usually referred to as sensory gating. On the one hand, an output sound signal generated by a hearing instrument can fundamentally interfere with this sensory gating, which can cause the body's own suppression of stress to be impaired. On the other hand, a hearing loss can lead to stress in a hearing-impaired person due to an increased level of hearing effort, in the same way as uncomfortably wearing a hearing instrument, e.g. as a result of pressure or even pain.

Especially in the case of hearing loss, there seems to be a bidirectional relationship between stress and hearing loss: acute (negative) stress perception can worsen hearing loss further; a hearing loss can lead to a level of stress that is no longer manageable for the affected person. A kind of circular self-amplification of the aforementioned effects can also set in.

As a specific measure for reducing the stress level of the wearer of the hearing instrument, by modifying an appropriate signal processing parameter a noise suppression (directional or single-channel) can preferably be increased, an emphasis of useful signals (e.g. speech by means of speech-specific measures) can be amplified, and/or level peaks, or generally a maximum loudness, can be reduced. In general, acoustic artifacts can also occur with the measures mentioned (in particular with noise suppression and the emphasis of useful signals), but in the case of a critical stress level, its reduction can be temporarily prioritized over a maximally realistic sound image.

For an application or as a specific measure of the signal processing, in particular the input signal can be analyzed for the presence of a speech signal in the ambient sound. In the presence of a speech signal, the at least one signal processing parameter is preferably adjusted to the predefined parameter value in such a way that a directivity of a directional microphone is amplified and/or an onset of speech segments is emphasized, and/or frequency bands with a high speech content are enhanced. This emphasizes the speech signal overall. On detecting the absence of a speech signal, a frequency-band-dependent gain and/or a frequency-band-dependent maximum output level are preferably lowered, and/or a settling time of a compression is shortened and/or a decay time of a compression is extended, and/or an applied strength of a noise reduction and/or a smoothing filter are increased. This generally reduces exposure to excessive sound levels and/or high level peaks as well as drastic level changes.

Advantageously, at a critical stress level, an applied strength and/or a notch depth of a tinnitus masking signal can be increased, e.g. by increasing the overall volume of a tinnitus masking signal, lowering a center frequency, and/or increasing a modulation depth. It is known that tinnitus often has a significant association with the stress level of the affected person, so that improved masking of the tinnitus can help to reduce the stress level.

If a heart rate and/or its rate of change and/or some other characteristic parameter of cardiovascular activity is acquired as a biometric parameter, a movement state of the wearer is preferably determined by means of an acceleration sensor and/or a motion sensor, and the determined movement state of the wearer is also used for the critical stress level. Thus, for example, an increase in the heart rate or the systolic or diastolic blood pressure as a result of movement can be detected so that such increases are not associated with a critical stress level, or a critical value for the heart rate or the relevant blood pressure can be set correspondingly higher with regard to the stress level as a result of energetic activity.

Advantageously, in addition to the characteristic parameter of the cardiovascular activity for the critical stress level, the detected ambient acoustic situation and/or a time of day can also be used. The time of day may be particularly relevant if the wearer regularly takes medications that affect the heart rate and/or blood pressure. A change in the heart rate or its rate of change can then be attributed according to the intake of the medication, and thus a critical value for the heart rate or blood pressure with regard to the stress level can be set accordingly higher. An ambient acoustic situation can be taken into account, for example, based on general empirical values.

In an advantageous embodiment, the presence of a specific acoustic ambient situation is detected from the plurality of different ambient acoustic situations based on the input signal, and when a critical stress level is detected in the wearer, in particular by a user input by means of an auxiliary device connected to the hearing instrument, such as a smartphone or a smartwatch, the critical stress level is assigned to the specific ambient acoustic situation and the analysis of the input signal is used to detect the presence of the specific acoustic ambient situation, and the detected acoustic ambient situation is used to infer the critical stress level of the wearer. This is a further means of identifying a critical stress level. The ambient acoustic situation can be used as the sole criterion for the critical stress level, or it can be weighted together with the heart rate or its rate of change.

Advantageously, a resting value for the characteristic parameter of the cardiovascular activity is first determined, the acquired characteristic parameter of the cardiovascular activity is compared with the corresponding resting value, and the critical stress level of the wearer is inferred from this. For example, in the case of a particularly high or low resting value, e.g. for a blood pressure, the heart rate or its rate of change, it must be assumed that a blood pressure, heart rate or its rate of change for a critical stress level is also increased or decreased accordingly.

Advantageously, the movement state of the wearer, which is determined by means of the acceleration sensor or the motion sensor, and/or a time of day and/or a characteristic parameter for an ambient sound, determined by means of a corresponding analysis of the input signal, are used for determining the resting value. The characteristic parameter for the ambient sound can be related in particular to unwanted noise, a classified listening situation, a speech component, the wearer's own voice, an overall volume, and/or a frequency characteristic of the ambient sound.

Movement, such as exercise, can increase the heart rate or blood pressure and shorten the exertion phase. The time of day can influence the heart rate or its rate of change, in particular for people who take regular medication (e.g. blood pressure reducers or similar), depending on the amount consumed. Loud noises, in particular unwanted noises, but also speech signals in general, in particular in noisy environments (e.g. “cocktail party” listening situation) and even hearing one's own voice, can generally increase the level of stress. The values for the characteristic parameter of cardiovascular activity determined in such time sequences are not used, or only to a reduced extent, when determining the respective resting value.

In an advantageous embodiment, a critical value for the characteristic parameter of the cardiovascular activity which corresponds to a critical stress level of the wearer is determined on the basis of a user input made by the wearer by means of a user interface, in particular via an auxiliary device connected to the hearing instrument such as a smartphone or a smartwatch, wherein the critical stress level is inferred at a later time in accordance with the critical value for the characteristic parameter of the cardiovascular activity, i.e. when a blood pressure or the heart rate or the rate of change of the heart rate reaches or exceeds the corresponding critical value. The user interface is preferably implemented in an auxiliary device, such as a smartphone or similar, that can be connected to the hearing instrument for data communication. In particular, this means that the wearer can signal via the user input that they are experiencing a critical stress level at the time in question, so that the corresponding value for blood pressure, heart rate or its rate of change can be used as an indicator of the critical stress level. The user input can be made, for example, via an appropriate app in the smartphone.

It proves to be further advantageous if the presence of a specific acoustic ambient situation from the plurality of different acoustic ambient situations is detected by means of the input signal; a hearing program, which is preassigned to the specific ambient acoustic situation and assigns a specific parameter value to the at least one parameter, is used for the signal processing of the input signal; a rating of the hearing program by the wearer is acquired on the basis of a user input by the wearer; when a critical stress level of the wearer is acquired, in the case of a negative rating of the hearing program the weighting of the negative rating is reduced in a user-assisted, automatic adjustment of the parameter value for the specific ambient acoustic situation, and/or in the case of a positive rating of the hearing program a sub-program comprising the parameter value is generated from the hearing program, which is applied to the specific ambient acoustic situation at a critical stress level of the wearer.

On the one hand, this allows negative ratings of hearing programs that are based solely or substantially on an acute perception of stress by the wearer to be filtered out for generating the parameter value for the signal processing. In addition, hearing programs that have received a positive rating at a critical stress level can be used when a critical stress level occurs again (i.e., used in later applications). As a result, from a particular hearing program a sub-program is deli fined which is preferably used when an ambient acoustic situation (“listening situation”) and a critical stress level are simultaneously present.

In particular, a hearing program is understood to mean the totality of parameters of the signal processing which are applied to an input signal in a certain ambient acoustic situation for generating an output signal.

The second object is achieved according to the invention by a hearing system comprising a hearing instrument, a sensor for acquiring a biometric parameter of a wearer of the hearing instrument, which gives information about a critical stress level of the wearer, and a control device, wherein the control device is designed to operate the hearing instrument in accordance with the biometric parameter for the critical stress level according to the method as claimed in any one of the previous claims.

The hearing aid system can in particular comprise an auxiliary device (e.g. a smartphone, a smartwatch or similar) that can be connected to the hearing instrument. In this case, the auxiliary device can comprise the control device and/or the or at least one sensor for acquiring said biometric parameter, and in particular means for acquiring a user input. The hearing instrument is preferably designed as a hearing aid. However, the control device may also be provided by a signal processing unit of the hearing instrument. The execution of the computation steps required for the method can also be carried out partly on a processor unit of the auxiliary device and partly on the signal processing unit of the hearing instrument. In the latter case, the control device is distributed over the hearing instrument and the auxiliary device.

The hearing aid system according to the invention shares the advantages of the method according to the invention. The advantages specified for the method and for its extensions can be transferred, mutatis mutandis, to the hearing aid system.

In the following an exemplary embodiment of the invention is explained in more detail based on drawings. In the drawings, schematically in each case:

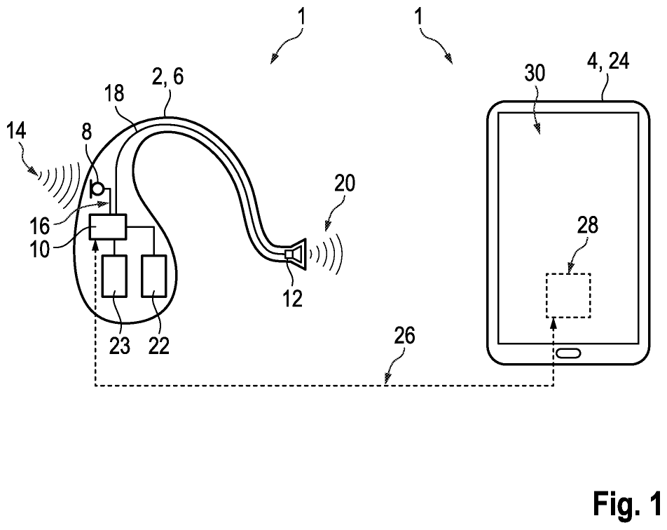

FIG. 1 shows a block diagram of a hearing aid system with a hearing aid and a smartphone, which is configured to detect a critical stress level of a user,

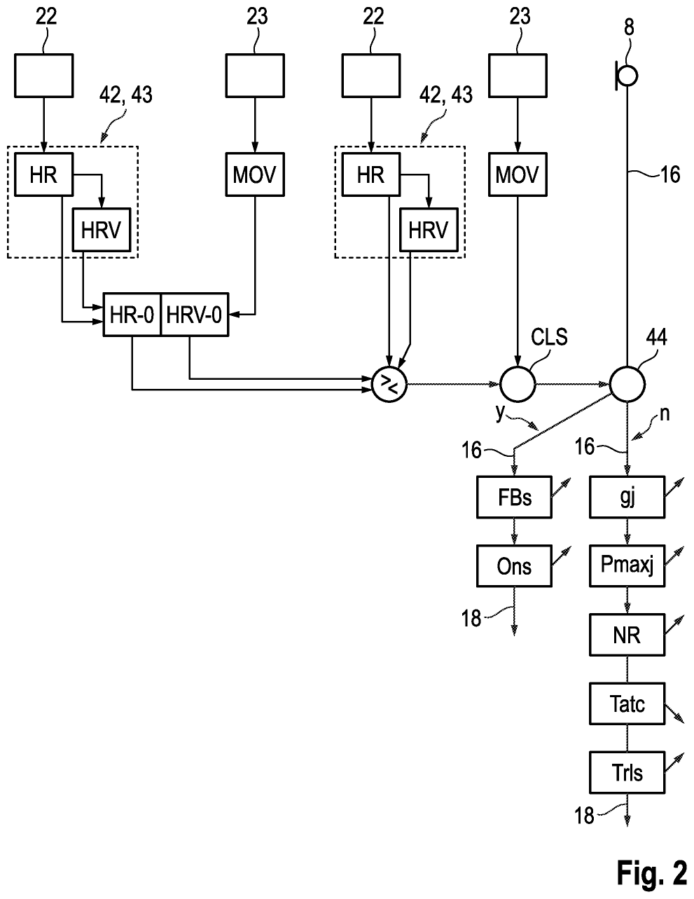

FIG. 2 shows a block diagram of a method for detecting a critical stress level of a hearing aid wearer by means of the hearing aid,

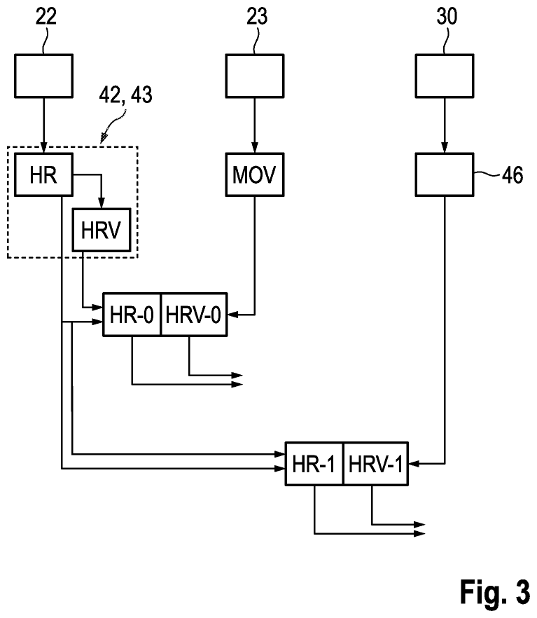

FIG. 3 shows a block diagram of an alternative embodiment of the method according to FIG. 2,

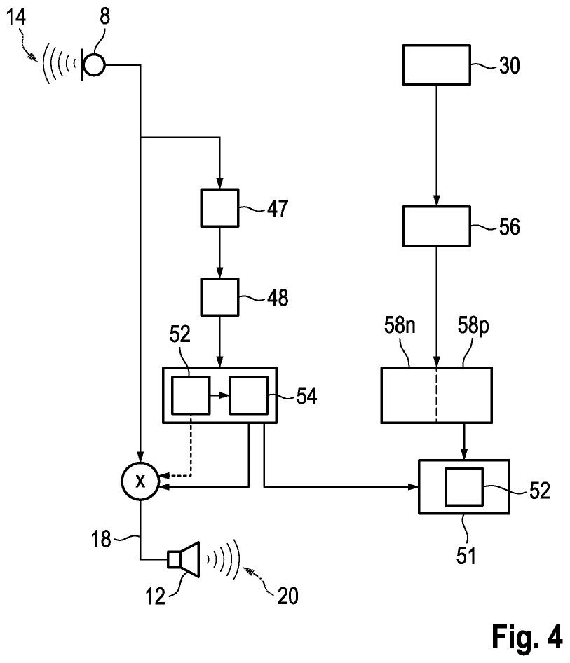

FIG. 4 shows a block diagram of a further alternative of the method according to FIGS. 2, and

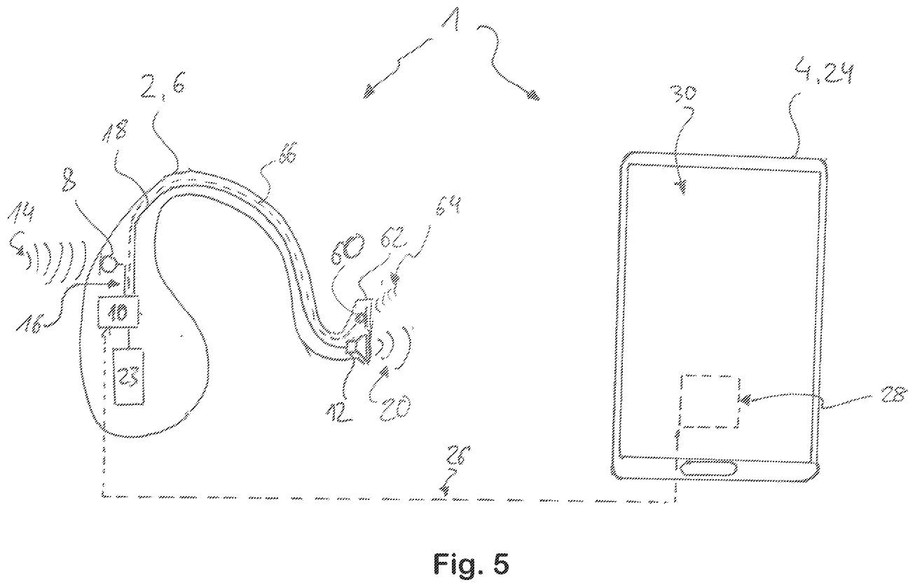

FIG. 5 shows a block circuit diagram of a hearing aid system with an alternative hearing aid to FIG. 1.

Equivalent parts and dimensions are provided with identical reference signs in all figures.

FIG. 1 shows a schematic block diagram of a hearing aid system 1 which comprises a hearing instrument 2 and an auxiliary device 4. The hearing instrument 2 in this case is designed as a hearing aid 6, and accordingly has a microphone 8, a signal processing unit 10 connected to the microphone 8, and a loudspeaker 12 connected to the signal processing unit 10. The hearing aid 6 can also comprise further microphones, not shown in FIG. 1, which are connected to the signal processing unit 10 in the same way. The microphone 8 is configured to generate an input signal 16 from an ambient sound 14. The input signal 16 (and optionally other input signals from microphones, not shown) is processed in the signal processing unit 10 to form an output signal 18, which is converted by the loudspeaker 12 into an output sound signal 20. In the processing of the input signal 16 in the signal processing unit 10, it is possible in particular to take a hearing loss of a wearer of the hearing aid 6 into account in such a way that the hearing loss is at least partially compensated by a frequency-band-dependent amplification and/or compression of the input signal 16.

Further, the hearing aid 6 has a PPG sensor 22, which is also connected to the signal processing unit 10. The PPG sensor 22 is configured to measure a heart rate of the wearer, so that a rate of change of the heart rate can also be determined in the signal processing unit 10 on the basis of the measured heart rate. Moreover, the hearing aid 6 has an acceleration sensor 23, which is also connected to the signal processing unit 10 and by means of which a change in movement of the wearer of the hearing aid 6 can be detected. By integrating the acceleration in the signal processing unit 10, a movement of the wearer can thus also be detected.

In a manner to be described below, at least one parameter of the signal processing of the input signal 16 carried out in the signal processing unit 10 is adjusted on the basis of the detected heart rate or its rate of change, in such a way that the corresponding output sound signal 20 is suitable for reducing a stress level of the wearer of the hearing aid 6. For this purpose, in particular the auxiliary device 4, implemented in the present case as a smartphone 24, is used, which can be connected to the hearing aid 6 via a Bluetooth connection 26. For this purpose, the hearing aid 6 and the smartphone 24 each have appropriately configured transmitting and receiving devices, not shown in detail in FIG. 1, (for example antennas) for establishing said Bluetooth connection 26. The smartphone 24 also has a processor unit 28 for the processing steps described below, as well as a touch screen 30, by means of which user inputs can be made by the wearer of the hearing aid 6 or the hearing system 1 using a corresponding application.

FIG. 2 shows a schematic block diagram of the sequence of a method by means of which the hearing instrument 2 according to FIG. 1 can be operated in accordance with a critical stress level of the wearer of the hearing instrument 2. First, during the operation of the hearing instrument in the manner described below, relationships between the stress perceived by the wearer of the hearing instrument 2 according to FIG. 1, biometric parameters relevant to this and parameters of the signal processing that can influence the stress level, are determined, and thus “learned” (shown in the left half of the picture). The said “learned” relationships relating to the biometric parameters are used at a later time (shown in the right half of the picture) for detecting an acute stress situation for the wearer of the hearing instrument 2, so that the parameters of the signal processing provided for this purpose are applied correspondingly to the input signal 16 in order to reduce the stress level by means of the output sound signal 20 generated from this input signal.

Biometric parameters 42 of the wearer of the hearing aid 6 are then determined using the PPG sensor 22. These biometric parameters 42 in this case are given by characteristic parameters 43 of a cardiovascular activity of the wearer, and include a heart rate HR of the wearer of the hearing aid 6, which is acquired by the PPG sensor 22, as well as a rate of change HRV of the heart rate HR, which is determined in the signal processing unit 10 from the heart rate HR. Furthermore, the characteristic parameters 43 of a cardiovascular activity, for example, can also include a systolic and/or diastolic blood pressure and/or a cardiac exertion phase. On the basis of the acceleration sensor 23, a movement activity MOV of the wearer of the hearing aid 6 is determined. The heart rate HR and its rate of change HRV are then compared with the determined movement activity MOV to determine resting values HR-0 and/or HRV-0, by using the values of the heart rate HR and its rate of change HRV in only those time sequences in which no significant movement activity MOV occurs. As already described above, a time of day (in particular when regular medication is taken) and/or a noise level or background can also be used for determining the resting values HR-0, HRV-0.

At a later time (right half of the image), the heart rate HR of the wearer is measured, also by means of the PPG sensor 22, and its rate of change HRV is determined accordingly. These two quantities are then compared with the corresponding resting values HR-0, HRV-0 which were previously determined. In the case of a deviation evaluated as critical (e.g. of one of the two biometric parameters 42 mentioned, or a predefined Cartesian deviation for the vector [HR, HRV]^{T} from the corresponding vector of the resting values, etc.), a check is first carried out as to whether a currently occurring movement activity MOV has been determined using the acceleration sensor 23 (in addition, an adjustment with regard to the time of day can also be carried out, see above). If this is not the case, a sufficiently high deviation, i.e. assessed as critical, of the heart rate HR or its rate of change HRV from their corresponding resting values is used to infer a critical stress level CSL.

In this case, that is, if a critical stress level is present, the input signal 16, which is generated by the microphone 8 of the hearing aid 6 from the ambient sound 14 at the current time, is subjected to a speech analysis 44 which checks whether the input signal 16 contains a speech signal. If this is not the case (path “n”), frequency-band-specific gain factors gj and frequency-band-specific maximum output levels Pmaxj are lowered, and noise reduction NR is increased. Furthermore, a settling time Tatc of a compression can be reduced and a decay time Trls of the compression can be increased. By the measures mentioned, the input signal 16 is processed to form the output signal 18 in such a way that the output sound signal 20 generated by the loudspeaker 12 is suitable for reducing the stress level of the wearer. In particular, the noise exposure for the wearer of the hearing aid 6 is reduced by the lowering of the frequency-band-specific gain factors gj, and the discomfort threshold, which can be reduced due to psychological factors under high levels of stress, is additionally avoided by lowering the maximum output level Pmaxj. Although greater use of noise reduction NR and/or a “sharper” use of compression may be more likely to cause acoustic artifacts, this is temporarily preferable in the present case to reduce the stress level of the wearer.

If a speech signal is present in the input signal 16 (path “y” of the speech analysis 44), those frequency bands FBs in the input signal 16 that contain a high speech component are relatively enhanced. The onset Ons of speech segments is also highlighted. In the case of multiple input signals (not shown) of the hearing aid 6, which are processed by the signal processing unit 10 by means of directional microphony, the directivity can also be increased. If a speech signal is present the aforementioned measures are an effective means of improving the speech intelligibility, and thereby of reducing the stress level of the wearer of the hearing aid 6 (even if a realistic auditory perception might be temporarily impaired).

The aforementioned measures, which are taken with regard to the processing of the input signal 16 either in the presence or absence of a speech signal and the simultaneous presence of the critical stress level CSL, can be taken in particular in the context of a specific hearing program (which may include additional signal processing measures depending on an ambient acoustic situation), which is specifically intended for the case of a critical stress level CSL in an ambient acoustic situation. On the other hand, the measures can also be carried out “ad hoc” and independently of a hearing program, so that the application is only carried out in accordance with the critical stress level CSL and, if necessary, the speech analysis 44. In general, the parameter in question is set to a parameter value such that a signal processing of the input signal 16 with the parameter value for the parameter is suitable for reducing the stress level, i.e. will lead to it in line with expectation and without unforeseeable exceptional circumstances.

If a critical stress level CSL is not determined, e.g. because the deviation of the heart rate HR or its rate of change HRV from the respective resting values HR-0, HRV-0 is not sufficiently large, or because a sufficiently large deviation can be attributed to a high level of movement activity MOV, the processing of the input signal 16 into the output signal 18 is preferably only carried out according to an ambient acoustic situation determined by means of the input signal 16 itself (“hearing situation”; not shown).

FIG. 3 shows a schematic block diagram of an alternative embodiment of that part of the method according to FIG. 2 which relates to “learning” the detection of the correct values of the biometric parameters 42 for the critical stress level CSL (left half of the picture in FIG. 2). As in the exemplary embodiment according to FIG. 2, the heart rate HR and its rate of change HRV are measured or determined as biometric parameters 42 and thus as characteristic parameters 43 of the cardiovascular activity of the wearer in order to determine the resting values HR-0, HRV-0 of the aforementioned biometric parameters 42 from a comparison with the movement activity MOV.

In addition, a user input 46 is acquired, which the wearer of the hearing aid 6 can enter in an appropriate app via the touch screen 30 of the smartphone 24. By the user input 46, the wearer signals to the hearing aid system 1 according to FIG. 1 that for him/herself the stress sensation is currently unpleasantly high. With regard to the heart rate HR and its rate of change HRV, this means that the values HR-1 and HRV-1 determined as critical by the user input 46 correspond to a critical stress level CSL. In the application not shown in FIG. 3 (cf. right half of the picture in FIG. 2), the critical stress level CSL can now be detected if the heart rate HR detected by means of the PPG sensor 22 (or its rate of change HRV) is sufficiently close to or above the value HR-1 (or HRV-1) determined for the critical stress level CSL. For values below HR-1 (or HRV-1), the resting value HR-0 (or HRV-0) for the range of values to be considered critical can be used.

FIG. 4 shows a schematic block diagram illustrating a further alternative to the method according to FIG. 2. In this case, the input signal 16, which is generated from the ambient sound 14 by the microphone 8 of the hearing aid 6 at a given time, is subjected to an analysis 47 in which an ambient acoustic situation 48 represented in the ambient sound 14 is detected. A hearing program 50 preassigned to the detected ambient acoustic situation 48 is then selected, which determines specific parameter values 52 for individual parameters 54 of the signal processing (such as the parameters mentioned in the description of FIG. 2, which are applied to the input signal 16). The input signal 16 is then processed according to the parameter values 52 defined by the hearing program 50 for the parameters 54, and the resulting output signal 18 is converted by the loudspeaker 12 into the output sound signal 20.

The wearer (not shown) of the hearing aid 6 now “hears” this output sound signal, and can make a positive or negative rating 58p or 58n of the hearing program 50 via a user input 56 (in a comparable way to the user input 46 according to FIG. 3). In addition, the wearer can report a subjectively perceived critical stress level CSL to the hearing aid system 1 via a user input 46. If such a report of a critical stress level CSL is made by the user input 46, according to the method the negative rating 58n of the hearing program 50 can give rise to a lower weighting of the associated parameter values 52 for a final adjustment of the hearing program 50, or also to a change of the parameter values 52 on the next occurrence of the ambient acoustic situation 48. However, if a positive rating 58p of the hearing program 50 is given instead, a sub-program 51 can also be generated on the basis of the parameter values 52 used, which is intended for a later application when the ambient acoustic situation 48 (to which the hearing program 50 is assigned) and the critical stress level CLS (preferably to be determined based on the biometric parameters 42) occur simultaneously.

Analogous to the exemplary embodiments according to FIGS. 2 and 3, the heart rate HR and its rate of change HRV, as well as optionally other characteristic parameters 43 of the cardiovascular activity than those shown in FIG. 2, can be additionally measured or determined (not shown) as biometric parameters 42. Furthermore, by comparison with the movement activity MOV it can be ensured that potentially increased values of the heart rate HR of its rate of change HRV are not the result of sporting activity or hard physical labor or similar, but are sufficiently informative for the stress level of the wearer. The resting values thus determined can be used at a later time (not shown) to detect a critical stress level in order to apply the sub-program 51 for the signal processing, which is assigned to the ambient acoustic situation 48 at the critical stress level (instead of the hearing program 50).

FIG. 5 shows a schematic block diagram of a hearing aid system 1 which comprises a hearing instrument 2 that forms an alternative to the hearing instrument 2 shown in FIG. 1. The hearing instrument 2 of FIG. 5 comprises an ear-canal microphone 60 instead of the PPG sensor 22 (in a further alternative variant, the hearing instrument may also comprise the PPG sensor 22 and the ear-canal microphone 60), which is arranged together with the loudspeaker 12 in an earpiece 62. The earpiece 62 must be at least partially inserted by the wearer (not shown) into an entrance of one of their two ear canals for the proper operation of the hearing instrument 2, so that the ear-canal microphone 60 is thereby directed into the ear canal, where it can receive an ear-canal sound 64 in the ear canal which is at least partially closed off by the earpiece 62.

The ear-canal microphone 60 generates an additional input signal 66 (dashed line) from the ear-canal sound 64, which is forwarded to the signal processing unit 10. From the ear-canal sound 64, the heart rate can be determined by means of the above processing in the additional input signal 66 by an appropriate analysis and/or filtering of the additional input signal 66. The said analysis or filtering of the additional input signal 66 can be carried out in particular in the signal processing unit 10. The ear-canal microphone 60 can in particular also perform a further functionality, for example as part of an occlusion suppression, which is preferably controlled by the signal processing unit 10.

The further processing of the heart rate determined by means of the additional input signal 66 proceeds as described above.

Although the invention has been illustrated and described in greater detail by means of the preferred exemplary embodiment, the invention is not restricted by the examples disclosed and other variations can be derived therefrom by the person skilled in the art without departing from the scope of protection of the invention.

### LIST OF REFERENCE SIGNS

1 hearing aid system
2 hearing instrument
4 auxiliary device
6 hearing aid
8 microphone
10 signal processing unit
12 loudspeaker
14 ambient sound
16 input signal
18 output signal
20 output acoustic signal
22 PPG sensor
23 acceleration sensor
24 smartphone
26 Bluetooth connection
28 processor unit
30 touchscreen
42 biometric parameters
43 characteristic parameter of cardiovascular activity
44 speech analysis
46 user input
47 analysis
48 ambient acoustic situation
50 hearing program
51 sub-program
52 parameter value
54 parameter (of signal processing)
56 user input
58n/p negative/positive rating
60 ear-canal microphone
62 earpiece
64 ear-canal sound
66 additional input signal
CSL critical stress level
FBs frequency bands with high speech content
gj frequency-band-dependent gain factor
HR heart rate
HR-0 resting value (of heart rate)
HR-1 (critical) value (of heart rate)
HRV rate of change (of heart rate)
HRV-0 resting value (of rate of change)
HRV-1 (critical) value (of rate of change)
MOV movement activity
n “No” path (of the speech analysis)
NR noise reduction
Ons onset of speech segments
Pmaxj frequency-band-dependent maximum output level
Tatc settling time (of a compression)
Trls decay time (of a compression)
y “Yes” path (of the speech analysis)

1-15. (canceled)

16. A method for operating a hearing instrument, the method comprising:
performing at least one of:
acquiring a biometric parameter of a wearer of the hearing instrument by a sensor; or
generating an electrical input signal from an ambient sound by at least one microphone of the hearing instrument, and detecting the presence of a specific ambient acoustic situation from a plurality of different ambient acoustic situations by an analysis of the input signal; and
determining a critical stress level of the wearer based on the acquired biometric parameter or the detected ambient acoustic situation; and
adjusting at least one parameter of a signal processing of the hearing instrument to a predefined parameter value based on the determined critical stress level.

17. The method according to claim 16, wherein the biometric parameter is a characteristic parameter of a cardiovascular activity of the wearer.

18. The method according to claim 17, wherein the acquired characteristic parameter of a cardiovascular activity of the wearer is at least one of a heart rate of the wearer, a rate of change of the heart rate of the wearer, a systolic blood pressure of the wearer, diastolic blood pressure of the wearer, or a duration of an exertion phase of the heart.

19. The method according to claim 18, wherein at least one of the heart rate, the systolic blood pressure, or diastolic blood pressure is measured using photoplethysmography.

20. The method according to claim 18, wherein at least one of the heart rate, the systolic blood pressure, or diastolic blood pressure is measured using an ear canal microphone, and the ear canal microphone is directed into the wearer's ear canal when the hearing instrument is worn in an intended position.

21. The method according to claim 16, further comprising determining a movement state of the wearer by an acceleration sensor and/or a motion sensor, and wherein the critical stress level of the wearer is additionally determined based on the movement state of the wearer.

22. The method according to claim 16, further comprising detecting the presence of a specific ambient acoustic situation by analyzing the input signal, and wherein the critical stress level of the wearer is additionally determined based on the detected ambient acoustic situation.

23. The method according to claim 21, further comprising:
determining a resting value for the characteristic parameter of the cardiovascular activity; and
comparing the acquired characteristic parameter of the cardiovascular activity with the corresponding resting value, and inferring the critical stress level of the wearer from the result.

24. The method according to claim 23, wherein the determining of the resting value is based at least partially on at least one of:
the movement state of the wearer, which is determined by the acceleration sensor or the motion sensor;
a time of day; or
a characteristic parameter for an ambient sound, which is determined based on a corresponding analysis of the input signal.

25. The method according to claim 17, further comprising determining a critical value for the characteristic parameter of the cardiovascular activity based on a user input performed by the wearer by way of a user interface, with the critical value corresponding to a critical stress level of the wearer, and the critical stress level being inferred at a later time in accordance with the critical value for the characteristic parameter of the cardiovascular activity.

26. The method according to claim 16, further comprising:
detecting the presence of a specific ambient acoustic situation from the plurality of different ambient acoustic situations by the input signal;
using a hearing program, which is preassigned to the specific ambient acoustic situation and assigns a specific parameter value to the at least one parameter, for the signal processing of the input signal;
acquiring a rating of the hearing program by the wearer based on a user input of the wearer; and
when a critical stress level of the wearer is detected
in the case of a negative rating of the hearing program, reducing the weighting of the negative rating during a user-assisted automatic adjustment of the parameter value for the specific ambient acoustic situation, and/or
in the case of a positive rating of the hearing program, generating a sub-program comprising the parameter value from the hearing program, which is applied to the specific ambient acoustic situation in the event of a critical stress level of the wearer.

27. The method according to claim 16, further comprising:
detecting the presence of a specific ambient acoustic situation from the plurality of different ambient acoustic situations by the input signal;
assigning, when a critical stress level of the wearer is detected, the critical stress level to the specific ambient acoustic situation; and
wherein the analysis of the input signal is used to detect the presence of the specific acoustic ambient situation, and the detected acoustic ambient situation is used to infer the critical stress level of the wearer.

28. The method according to claim 16, further comprising:
analyzing the input signal for the presence of a speech signal in the ambient sound;
when a speech signal is present:
setting the at least one parameter of the signal processing to the predefined parameter value in order to at least one of:
amplify a directivity of a directional microphone,
emphasize an onset of speech segments, or
enhance frequency bands with a high speech content;
when a speech signal is not present:
setting the signal processing to the predefined parameter value in order to at least one of:
reduce a frequency-band-dependent gain and/or a frequency-band-dependent maximum output level,
shorten a settling time of a compression and/or extend a decay time of the compression is extended, or
increase an applied strength of a noise reduction and/or a smoothing filter.

29. The method according to claim 16, wherein at a critical stress level, an applied strength and/or a notch depth of a tinnitus masking signal is increased.

30. A hearing aid system, comprising:
a hearing instrument;
a sensor for detecting a biometric parameter of a wearer of the hearing instrument, said biometric parameter of the wearer being configured to provide information about a critical stress level of the wearer; and
a control device being configured to operate the hearing instrument based at least in part on the biometric parameter for the critical stress level according to the method of claim 16.
